(19) United States
(12) Patent Application Publication
(10) Pub. No.: US 2012/0149765 A1
(43) Pub. Date: Jun. 14, 2012

(54) SGK1 AS DIAGNOSTIC AND THERAPEUTIC TARGET

(76) Inventor: Florian Lang, Tuebingen (DE)

(21) Appl. No.: 13/315,850

(22) Filed: Dec. 9, 2011

Related U.S. Application Data
(63) Continuation of application No. 10/547,746, filed on Sep. 26, 2006, now abandoned.

(51) Int. Cl. A61K 31/7088; C12Q 1/68; C07K 16/40; A61K 31/473; A61P 9/12; G01N 33/566; A61K 31/553

(52) U.S. Cl. 514/44 R; 435/7.1; 435/6.11; 514/211.08; 435/6.12; 514/280; 530/389.8

(57) ABSTRACT

The invention relates to the utilization of a substance for diagnostic determination of sgk1 (serum and glucocorticoid dependent kinase 1) and to the utilization of an active agent in order to influence sgk1 for therapeutic treatment of diseases associated with disordered activity of the tissue factor and to a diagnostic kit related to such substances or such active agents.

The present invention relates to the use of a substance for diagnostically detecting sgk1 (serum and glucocorticoid-dependent kinase 1) and to the use of an active compound for influencing sgk1 for the therapeutic treatment of diseases which are connected with a disturbed activity of TF (tissue factor), as well as to a diagnostic kit which is related thereto.

A large number of external signals to which a cell is subjected in its environment lead to intracellular phosphorylation/dephosphorylation cascades for the purpose of enabling these signals to be transferred rapidly and reversibly from the plasma membrane and its receptors into the cytoplasm and the cell nucleus. It is only the regulation of individual proteins which are involved in these cascades which makes possible the high degree of specificity and flexibility of the cells, which specificity and flexibility in turn enable the cells to react very rapidly to extracellular signals. It is, in particular, kinases, i.e. proteins which transfer a phosphate group to individual substrates, which are involved in these regulation processes. The serum and glucocorticoid-dependent kinase (sgk) was originally cloned from rat mammary carcinoma cells (Webster M K, Goya L, Firestone G L. J. Biol. Chem. 268 (16): 11482-11485, 1993; Webster M K, Goya L, Ge Y, Maiyar A C, Firestone G L. Mol. Cell. Biol. 13 (4): 2031-2040, 1993). The human kinase hsgk was cloned from liver cells as a cell volume-regulated gene (Waldegger S, Barth P, Raber G, Lang F. Proc. Natl. Acad. Sci. USA 94: 4440-4445, 1997). It was found that the rat kinase (Chen S Y, Bhargava A, Mastroberardino L, Meijer O C, Wang J, Buse P, Firestone G L, Verrey F, Pearce D. Proc. Natl. Acad. Sci. USA 96: 2514-2519, 1999; Naray-Fejes-Toth A, Canessa C, Cleaveland E S, Aldrich G, Fejes-Toth G. J. Biol. Chem. 274: 16973-16978, 1999) stimulates the epithelial Na⁺ channel (ENaC). It was furthermore shown that an increase in the activity of the ENaC is accompanied by hypertension (Warnock D G. Kidney Ind. 53 (1): 1824, 1998).

It was shown in DE 197 08 173 that hsgk1 possesses considerable diagnostic potential in connection with many diseases, such as hypernatremia, hyponatremia, diabetes mellitus, renal insufficiency, hypercatabolism, hepatic encephalopathy and microbial or viral infections, which are influenced patho-physiologically by a change in cell volume.

DE 199 17 990 describes kinase inhibitors, such as staurosporine, chelerythrine or transdominantly inhibitory kinase, which can be employed in the therapy of cell volume-dependent diseases.

hsgk is also expressed in the brain (Waldegger S, Barth P, Raber G, Lang F. Proc. Natl. Acad. Sci. USA 94: 4440-4445, 1997), where it regulates the Kv1.3 voltage-dependent K⁺ channels. It was shown that these K⁺ channels of the Kv1.3 type are involved in regulating neuronal excitability (Pongs O. Physiol. Rev. 72: 69-88, 1992), in regulating cell proliferation (Cahalan M D and Chandy K G, Cur. Opin. Biotech. 8 (6): 749-756, 1997) and in regulating apoptotic cell death (Szabo I, Gulbins E, Apfel H, Zhan X, Barth P, Busch A E, Schlottmann K, Pongs O, Lang F. J. Biol. Chem. 271: 20465-20469, 1999; Lang F, Szabo I, Lepple-Wienhues A, Siemen D, Gulbins E. News Physiol. Sci. 14: 194-200, 1999). Kv1.3 is also important in regulating lymphocyte proliferation and function (Cahalan M D and Chandy K G, Cur. Opin. Biotech. 8 (6): 749-756, 1997). Two further members of the sgk family, i.e. sgk2 and sgk3, have been cloned (Kobayashi T, Deak M, Morrice N, Cohen P. Biochem. J. 344: 189-197, 1999). Furthermore, it has been found that the sgks form a serine-threonine protein kinase family which can be regulated transcriptionally and posttranscriptionally. Like sgk1, sgk2 and sgk3 are also activated by, for example, insulin and IGF1 by way of the P13 kinase pathway. However, the sgk protein family has not thus far been completely characterized.

Accordingly, the invention is based on the object of utilizing sgk1 for novel diagnostic and therapeutic applications.

Surprisingly, it has been possible to demonstrate that overexpressing intact sgk1, as compared with expressing inactive sgk1, leads to an increase in coagulant activity. In this experimental system, the coagulation was triggered by the tissue factor (TF). TF is a 47 kDa transmembrane glycoprotein which serves as a primary connecting link between vascular cells or mononuclear cells and the hemostatic system. In acting in this way, TF initiates the blood coagulation cascade (Davie E W, Fujikawa K, Kisiel W. Biochemistry 30: 10363-10370, 1991). TF initiates blood coagulation by binding with high affinity to factors VII/VIIa. The resulting complex initiates the activation of factors IX and X, with this being followed by the generation of thrombin. Thrombin, for its part, catalyzes the conversion of fibrinogen into fibrin, leading to fibrin deposition and blood coagulation (Nemerson Y. Blood 71: 1-8, 1998).

An increase in the expression of TF is not necessarily associated with an increase in the biological activity of TF. Functionally active TF depends on the expression of a biologically active form at the cell surface. In vascular smooth muscle cells (SMCs) and monocytes, only 10-20% of the total cellular TF, which also constitutes the biologically active form, is available at the cell surface while the remaining TF is present in intracellular pools (approximately 30%) and as latent surface TF (50-60%) (Preissner K T, Nawroth P P, Kanse S M. J. Pathol. 190: 360-372, 2000; Schecter A D, Giesen P L, Taby O, Rosenfield C L, Rossikhina M, Fyfe B S, Kohtz D S, Fallon J T, Nemerson Y, Taubmann MB. J. Clin. Invest: 100: 2276-2285, 1997). It has been shown that, in addition to its coagulant effect (Ruef J, Hu Z Y, Yin L Y, Wu Y, Hanson S R, Kelly A B, Harker L A, Rao G N, Runge M S, Patterson, C. Circ. Res.: 24-33, 1997), TF also plays an important role in the metastasis of tumors and in angiogenesis (Lwaleed B A and Cooper A J. Medical Hypotheses. 55: 470-473, 2000; Verheul H M W, Jorna A S, Hoekman K, Broxterman H J, Gebbink M F B G, Pinedo H M. Blood: 4216-4221, 2000). In this way, the functional data which have been found show that the effects of sgk1 are suitable for influencing the expression and/or function of TF at the cell membrane and thus for indirectly influencing the coagulability of the blood, the adherence of tumor cells, with subsequent metastasis, and angiogenesis as well as diseases in which angiogenesis plays a role. Stimulation of sgk1 leads to an increase in the expression of tissue factor while inhibition of sgk1 leads to a decrease in the expression of active tissue factor, and it is thereby possible to influence the above-described indications indirectly in a stimulatory or inhibitory manner.

Consequently, the object according to the invention is achieved by the subject matter of the independent claims 1, 6, 7, 22, 26 and 28. Preferred embodiments are specified in the dependent claims. The content of all these claims is hereby incorporated by reference into the description.

According to the invention, at least one substance can be used for detecting the expression and/or function of sgk1 in eukaryotic cells. This thereby also makes it possible, in particular, to diagnose diseases which are connected with a disturbed activity of TF. This substance could, for example, be an antibody which is directed against Sgk1 and which can be employed in a detection method, such as ELISA (enzyme-linked-immuno sorbent assay), which is known to the skilled person. In such immunoassays, the specific antibody (or homologous test antigens in the case of antibody determinations) which is directed against the antigen to be determined (Sgk1) is bound to a support substance (e.g. cellulose or polystyrene), on which immune complexes are formed following incubation with the sample. In a subsequent step, a labeled antibody is added to these immune complexes. The immune complex-bound enzyme-substrate complexes can be visualized by adding a chromogenic substrate to the reaction mixture, or the antigen concentration in the sample can be determined, by way of a photometric determination of the immune complex-bound marker enzymes, by comparing with standards of known enzyme activity.

Other substances which can be used for the diagnostic detection are oligonucleotides, which are suitable, using the polymerase chain reaction (PCR), for providing a quantitative detection of sgk1 by means of a molecular genetic method in which selectively determined DNA segments are amplified.

In another preferred embodiment, the substances employed in the use according to the invention are polynucleotides which can hybridize with sgk1 under stringent conditions. These polynucleotides can be used, for example, to carry out Southern or Northern blots in order, in this way, to determine the DNA or RNA content of sgk1. The skilled person is familiar with appropriate methods. The transcription rate of sgk1 can, for example, be analyzed in this way.

In a particularly preferred embodiment of the use according to the invention, the substance, that is, in particular, antibodies, oligonucleotides and/or polynucleotides, is suitable for detecting mutations in sgk1. Interestingly, it has been found that certain mutations in sgk1 are associated with an increase in the expression and/or activity of the kinase. This was observed, in particular, in the case of two nucleotide polymorphisms (SNPs). These nucleotide polymorphisms are located in intron 6 (T→C), in the first place, and in exon 8 (C→T) in human sgk1. In this connection, the reader is referred to WO 02/074987, in which it is shown that these nucleotide polymorphisms are connected with a genetic predisposition to hypertension. Similar findings have also been made in the case of other mutations, in particular insertion mutations. The invention therefore envisages using appropriate antibodies, oligonucleotides and/or polynucleotides to detect corresponding mutations which are connected with an increase in the expression and/or activity of Sgk1 and, in this way, to be able to draw conclusions for the diagnosis of diseases which are connected with a disturbed activity of TF. The skilled person is familiar with the methodological approach employed in the described uses. Other methods with which the expression and/or function of sgk1 can be detected quantitatively will be evident to the skilled person and are likewise encompassed by the invention.

The invention claims an active compound for influencing, in particular inhibiting or activating, the expression and/or function of sgk1 in eukaryotic cells, for the purpose of treating diseases which are connected with a disturbed activity of TF. Since sgk1, like sgk2 and sgk3 as well, is a kinase, kinase inhibitors which are known to the skilled person, such as staurosporine, chelerythrine, etc., in particular, as well as other substances such as transdominantly negative kinase mutants, come into consideration. The skilled person is familiar with these substances and the substances can be obtained from commercial (Sigma, Calbiochem, etc.) as well as noncommercial sources. Examples of activators which can be used are recombinantly altered mutants of sgk1 as well as inhibitors of phosphatases, for example. The skilled person is also familiar with phosphatase inhibitors and some of them are likewise commercially (Sigma, Calbiochem, etc.) as well as noncommercially available. Using phosphatase inhibitors would inhibit dephosphorylation and, as a result, the sgk1-activated target (TF) would remain in the activated state. Preference is given to using these active compounds for producing a medicament or a pharmaceutical composition.

In another preferred embodiment of the invention, the active compound is directed against skg1 itself. The active compounds can, for example, be antisense sequences, what are termed kinase deficient mutants, or else kinase inhibitors such as the staurosporine and/or chelerythrine, or their analogs, which have already been mentioned above. The active compound can furthermore also be a so-called small molecular compound or a polynucleotide which encodes a peptide which influences, preferably inhibits or activates, the expression of sgk1.

In another preferred embodiment of the invention, the active compound is directed against activators, inhibitors, regulators and/or biological precursors of sgk1. These activators, inhibitors, regulators and/or biological precursors could be members of the sgk1 signal transduction cascade which are located upstream and/or downstream, transcription factors which are responsible for the level of expression of sgk1, proteases which [lacuna] for the proteolytic breakdown of activators, inhibitors, regulators and/or biological precursors of sgk1, or else thus far unknown molecules which are influenced by the active compound and are involved in the expression and/or function of sgk1.

According to the invention, it is possible to use known active compounds as well as active compounds which are still unknown. In a particularly preferred embodiment, the active compound which is directed against activators, inhibitors, regulators and/or biological precursors of sgk1 is what is termed a small molecular compound, in particular a compound of this nature having a molecular weight (MW) of <1,000. Small molecular compounds can, for example, be kinase inhibitors such as the imidazole derivatives SB 203580 (MW 377.4) or SB 202190 (MW 331.3), both of which are known inhibitors of kinase expression and marketed commercially by Calbiochem.

The invention can be used for treating all forms of diseases which are connected with disturbed activity of TF. Coagulopathies and/or angiopathies of the inherited or acquired type come into particular consideration in this connection. Coagulopathies are understood as meaning coagulation disturbances in general. Examples of inherited coagulopathies (what are termed defect coagulopathies) are dysfibrinogenemia, hypoproconvertinemia, hemophilia B, Stuart-Prower defect, etc. Examples of acquired coagulation disturbances are prothrombin complex deficiency, consumption coagulopathy, hyperfibrinolysis, immuno-coagulopathy and also complex coagulopathies. Both forms of coagulopathy are caused by a deficiency or functional disturbance of a variety of plasma coagulation factors. In conformity with the differing symptomatology, a distinction is made between coagulopathies having a hemorrhagic tendency (minus coagulopathies) and coagulopathies having a thrombosis tendency (plus coagulopathies) as well as hepatogenic, cardiogenic and immune coagulopathies, corresponding to the site of the cause. Consequently, by activating or inhibiting sgk1, the disposition of the blood to coagulate can be decreased or increased and thereby adapted to the medical indication. Similar considerations also apply to angiopathies, i.e. diseases which are brought together under the generic term of vascular diseases, such as diabetic angiopathy, diabetic microangiopathy, pulmonary hypertension, arteriosclerosis, etc. In this case, too, the active compound can be used, in particular, for treating inherited and/or acquired angiopathies.

In particularly preferred embodiments, use is made of a substance for detecting, or of an active compound for treating, pulmonary hypertension and/or arteriosclerosis.

In another preferred embodiment, the active compound is used for stimulating or inhibiting angiogenesis. Angiogenesis is understood as being the development of blood vessel walls, e.g. during embryonic development, and a number of angiogenesis-dependent diseases are known to the skilled person, for example diabetes mellitus, tumorigenesis and autoimmune diseases. In another preferred embodiment, the active compound is used for stimulating or inhibiting wound healing.

The invention also relates to a diagnostic kit. This kit comprises at least one substance which is suitable for detecting the expression and/or function of skg1, for the purpose of diagnosing diseases which are connected to a disturbed activity of TF. The diagnostic kit according to the invention is characterized, in particular, in that the substances used for detecting the expression and/or function of sgk1 are antibodies directed against Sgk1, oligonucleotides for a polymerase chain reaction for amplifying DNA segments of sgk1 and/or polynucleotides which are able to hybridize with sgk1 under stringent conditions. In this connection, very particular preference is given to using these substances to detect mutations, in particular nucleotide polymorphisms and/or insertion (mutations) which are associated with an increase in the expression and/or activity of sgk1. In this regard, the reader is referred to the above description.

It is furthermore possible to use such a kit to diagnose diseases which are associated with an overexpression or underexpression or hyperfunction or hypofunction of sgk1. These diagnostic agents can be used selectively in a diagnostic kit in order, inter alia, to detect diseases such as the above-described coagulopathies, angiopathies, angiogenesis-dependent diseases, diseases of wound healing, etc. In this connection, too, the diseases can be detected by detecting a disturbed expression and/or function of sgk1. In particular, this substance can be a substance which provides this detection on the nucleotide and/or peptide level or polynucleotide and/or polypeptide level. With regard to the additional features of such a substance, the reader is referred to the appropriate preceding text in the description.

In addition to this, the invention encompasses a method for diagnosing diseases which are connected with a disturbed activity of TF. In this context, the expression and/or function or activity of sgk1 is detected quantitatively in a body sample taken from a patient. This body sample can, for example, be a fluid such as blood or urine or else, for example, a cell sample. The quantitative detection is, for example, effected using antibodies directed against sgk1, using oligonucleotides which are suitable for a polymerase chain reaction for amplifying DNA segments of sgk1 and/or using polynucleotides which are able to hybridize with DNA and/or mRNA of sgk1 under stringent conditions. In this method, particular preference is given to using said substances for detecting particular mutations, in particular nucleotide morphisms and/or insertions, in sgk1, with these particular mutations being connected with an increase in the expression and/or function or activity of sgk1. The diseases to be diagnosed are, for example, diseases which are connected with disturbed blood coagulation or vascular diseases such as pulmonary hypertension and arteriosclerosis.

The invention furthermore encompasses a pharmaceutical composition which comprises at least one active compound, which influences, in particular inhibits or activates, the expression and/or function of sgk1, and preferably, where appropriate, a pharmaceutical excipient. In this connection, the active compound can be a kinase inhibitor such as the inhibitors staurosporine, chelerythrine, SB 203580 and SB 202190, or their analogs, which have already been mentioned above, or else other substances. The active compound can furthermore be a polynucleotide which encodes a peptide, preferably a polypeptide, with this peptide influencing, preferably inhibiting or activating, the expression of sgk1. An example of a polypeptide according to the invention is what is termed a kinase deficient mutant. Other examples of how the expression and/or function can be influenced by way of recombinantly altered variants of the target protein are familiar to the skilled person and can be found in a number of textbooks/reference books as well as instructions for laboratory work (e.g. Maniatis T, Fritsch E F, Sambrook J. Cold Spring Harbor, N.Y.: Cold Spring Harbor Laborator, 1996; Leonard G, Davis PhD, Michael W, Kuehl M d, James F, Battey M D. McGraw-Hill Professional Publishing, 1995). The active compound according to the invention can furthermore be what is termed a small molecular compound, preferably a small molecular compound having a molecular weight (MW) of <1,000. Furthermore, the active compound can also be an antisense sequence, i.e. a sequence which is able to form a double strand duplex with the mRNA and thereby inhibit the translation of a target polypeptide. It is also possible to use the sequence of sgk1 itself in order to achieve overexpression, for example by incorporating the sequence into vectors or plasmids, with it also being possible to modify the target sequence beforehand with “carrier” molecules, e.g. promoters. With regard to additional features of such a composition, the reader is referred to the appropriate previous text in the description.

Finally, the invention encompasses a pharmaceutical composition which comprises an effective quantity of at least one active compound which influences, in particular inhibits or activates, the expression and/or function of activators, inhibitors, regulators and/or biological precursors of sgk1. This pharmaceutical composition can, where appropriate, preferably also comprise a pharmaceutical excipient. These activators, inhibitors, regulators and/or biological precursors of sgk1 can, for example, be other kinases which are involved in the regulation of the activity of sgk1, transcription factors which play a role for the level at which sgk1 is expressed, as well as other known members, or members which are thus far unknown, of sgk1 in a transduction cascade, as well as the molecules which have already been described above. Polynucleotides which encode a peptide which influences, preferably inhibits or activates, the expression of activators, inhibitors, regulators and/or biological precursors of sgk1 can also be present in such a composition. It is also possible to use small molecular compounds which preferably have a molecular weight (MW) of <1,000 and which are directed against activators, inhibitors, regulators and/or biological precursors of sgk1 and which, in this connection, inhibit or activate the expression and/or function of this kinase. With regard to additional features of such an active compound, the reader is referred to the appropriate previous text in the description.

The existing, and additional, features of the invention ensue from the following description of preferred embodiments in combination with the subclaims and the figures. In this connection, the individual features can in each case be realized on their own, or several features can be realized in combination with each other.

The figures show the following:

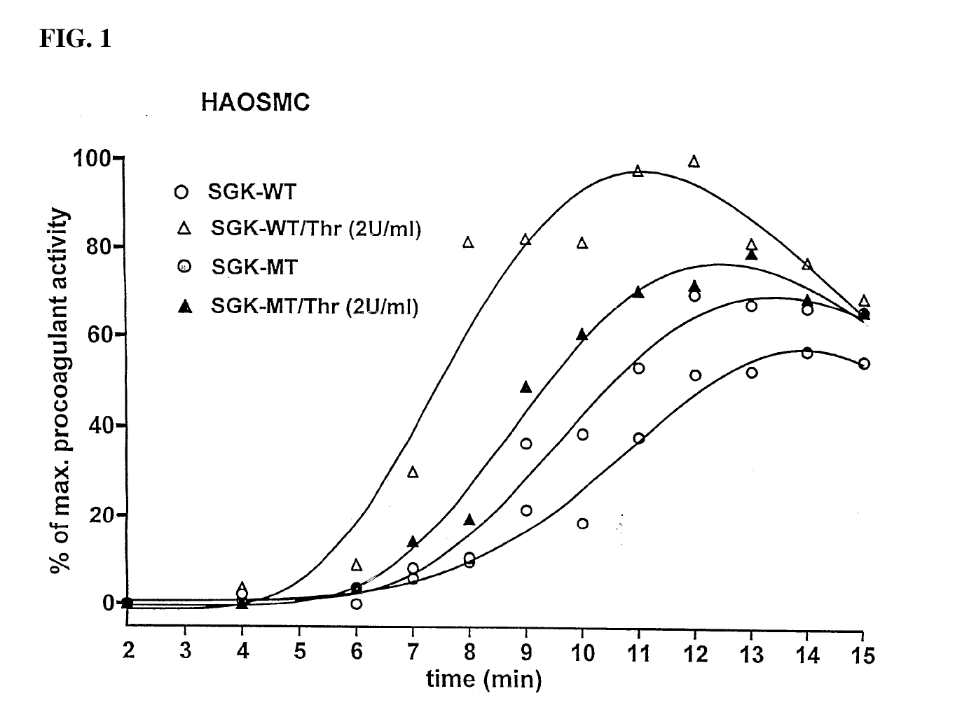

FIG. 1: Stimulation of the procoagulant activity of vascular smooth muscle cells.

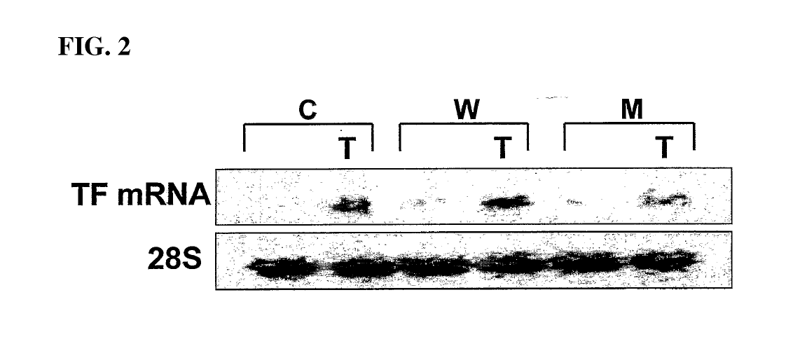

FIG. 2: Regulation of tissue factor SGK in human vascular smooth muscle cells (Northern blot). T: thrombin (3 U/ml) 4 h, C: control, W: SGK wild-type, M: SGK mutant.

### EXPERIMENT

In FIG. 1, the procoagulant activity, in % of the maximum value, is plotted against the time after recalcification. In the experiment relating to FIG. 1, the procoagulant activity of human vascular smooth muscle cells (HAOSMC) was measured by measuring thrombin formation during the coagulation process in recalcified blood platelet-poor plasma (PPP) (Beguin S, Lidhout T, Hemker H C: Throm. Haemost. 61: 25-29, 1998). For this, confluent vascular smooth muscle cells were kept for 24 hours in serum-free medium, then washed three times in HEPES-tyrode solution, and then incubated with human PPP. The formation of thrombin was induced by adding 16.7 mM CaCl₂ to the incubation medium. In each case 20 μl of the supernatant were removed every one to two minutes and the formation of thrombin in the removed volume was determined using the dye S-2238 (Haemochrom Diagnostica). The optical density was determined at 405 nm in a spectrophotometer (Uvikon, Contron-instruments). The dependence of the surface procoagulant activity of smooth muscle cells on the availability of the membrane-bound tissue factor was demonstrated using neutralizing antibodies directed against human tissue factor (Mab#4508; American Diagnostica; 10 μg/ml 20 minutes before recalcifying the PPP).

As FIG. 1 shows, the procoagulant activity of human vascular smooth muscle cells increases a few minutes after adding CaCl₂. This increase is slower when the inactive kinase (sgk-MT) is expressed than when the normal kinase (sgk-WT) is expressed. The additional administration of thrombin (Thr) leads, as expected, to the increase in procoagulant activity being accelerated. In this connection too, the effect is more pronounced and more rapid in cells which are expressing the intact kinase than in cells which are expressing an inactive mutant. Cells which are expressing intact (wild-type) sgk kinase (sgk-WT) exhibit, at each time point, a higher procoagulant activity than do cells which are expressing an inactive sgk mutant (sgk-MT), irrespective of whether thrombin has been added (sgk-WT/Thr and, respectively, Sgk-MT/Thr) or not.

This result demonstrates unambiguously that overexpression of intact sgk1 in vascular smooth muscle cells leads to an increase in coagulant activity. As a result of the known important role of TF in a variety of cell processes, this result also demonstrates that hyperactivity of sgk1 in association with an increase in expression of tissue factor at the cell membrane can promote the coagulability of the blood, make possible the adherence of tumor cells, with subsequent metastasis, and increase angiogenesis. Conversely, this mechanism would be suppressed by suppressing sgk1 expression or by pharmacologically inhibiting sgk1.

FIG. 2 shows a Northern blot of tissue factor mRNA (TF mRNA) from control cells harboring a control plasmid (C: control), cells containing transfected active kinase (W: SGK wild-type) and cells containing transfected inactive kinase (M: SGK mutant). The cells are human vascular smooth muscle cells equivalent to the cells used in the above experiment. 28S rRNA is loaded on as the internal standard. Human TF cDNA was used as the probe. The cells were in each case treated without and with thrombin (3 U/ml) for 4 hours. It can be seen from the Northern blot that the transcription of the tissue factor mRNA is upregulated on thrombin treatment in the cells containing active SGK (W) as compared with the control cells, whereas transcription is reduced in the cells containing inactive SGK mutant. This shows clearly that the expression of tissue factor is regulated by SGK in human vascular smooth muscle cells.

1-34. (canceled)

35. The method of diagnosing diseases which are connected with a disturbed activity of TF (tissue factor) in which the expression or function of sgk1 in eukaryotic cells is detected by at least one substance.

36. The method as claimed in claim 35, wherein said substance is an antibody which is directed against sgk1.

37. The method as claimed in claim 35, wherein said substance is an oligonucleotide which can be used to amplify particular DNA segments of sgk1 in a polymerase chain reaction (PCR).

38. The method as claimed in claim 35, wherein said substance is a polynucleotide which can hybridize with sgk1 under stringent conditions.

39. The method as claimed in claim 35, wherein said substance is suitable for detecting mutations, in particular nucleotide polymorphisms (SNPs) or insertions, in sgk1.

40. The method of treating diseases which are connected with a disturbed activity of TF in which the expression or function of sgk1 in eukaryotic cells is influenced, in particular inhibited or activated by at least one active compound.

41. The method as claimed in claim 40, wherein said active compound is directed against sgk1.

42. The method as claimed in claim 40, wherein said active compound is directed against activators, inhibitors, regulators or biological precursors of sgk1.

43. The method as claimed in claim 40, wherein said active compound is a kinase inhibitor, preferably staurosporine or chelerythrine or one of their analogs.

44. The method as claimed in claim 40, wherein said active compound is a polynucleotide which encodes a peptide, preferably a polypeptide, with this peptide influencing, preferably inhibiting or activating, the expression and/or function of sgk1.

45. The method as claimed in claim 40, wherein said active compound is a small molecular compound, preferably a small molecular compound having a molecular weight (MW) of <1.000.

46. The method as claimed in claim 35, wherein said diseases which are connected with a disturbed activity of TF are coagulopathies.

47. The method as claimed in claim 46, wherein said coagulopathies are inherited coagulopathies.

48. The method as claimed in claim 46, wherein said coagulopathies are acquired coagulopathies.

49. The method as claimed in claim 35, wherein said diseases which are connected with a disturbed activity of TF are angiopathies.

50. The method as claimed in claim 49, wherein said angiopathies are inherited angiopathies.

51. The method as claimed in claim 49, wherein said angiopathies are acquired angiopathies.

52. The method as claimed in claim 49, wherein said diseases are pulmonary hypertension or arteriosclerosis.

53. The method as claimed in claim 40, wherein said active compound is used for stimulating or inhibiting angiogenesis.

54. The method as claimed in claim 40, wherein said active compound is used for stimulating or inhibiting wound healing.

55. A diagnostic kit comprising at least one substance for detecting the expression or function of sgk1 for the purpose of diagnosing diseases which are connected with a disturbed activity of TF.

56. The diagnostic kit as claimed in claim 55, wherein said substance is an antibody which is directed against sgk1.

57. The diagnostic kit as claimed in claim 55 for the purpose of diagnosing diseases which are associated with hyperfunction or hypofunction of sgk1.

58. The diagnostic kit as claimed in claim 58 for the purpose of diagnosing coagulopathies, angiopathies or angiogenesis-dependent diseases.

59. A method for diagnosing diseases which are connected with a disturbed activity of TF, with the expression or function of sgk1 being detected quantitatively, in a body sample taken from a patient, using antibodies directed against sgk1, using oligonucleotides with which particular DNA segments of sgk1 can be amplified in a polymerase chain reaction (PCR) or using polynucleotides which are able to hybridize with sgk1 DNA or mRNA under stringent conditions.

60. The method as claimed in claim 59, wherein said antibodies, oligonucleotides or polynucleotides are used to detect particular mutations, in particular nucleotide polymorphisms or insertions, in sgk1.

61. A pharmaceutical composition, comprising an effective quantity of at least one active compound, which influences, in particular inhibits or activates, the expression or function of sgk1, and, where appropriate, a pharmaceutical excipient.

62. The pharmaceutical composition as claimed in claim 61, wherein said active compound is a kinase inhibitor, preferably staurosporine or chelerythrine or one of their analogs.

63. The pharmaceutical composition as claimed in claim 61, wherein said active compound is a polynucleotide which encodes a peptide, preferably a polypeptide, with this peptide influencing, preferably inhibiting or activating, the expression or function of sgk1.

64. The pharmaceutical composition as claimed in claim 61, wherein said active compound is a small molecular compound, preferably a small molecular compound having a molecular weight (MW) of <1.000.

65. The pharmaceutical composition as claimed in claim 61, comprising an effective quantity of at least one active compound, which influences, in particular inhibits or activates, the expression or function of activators, inhibitors, regulators or biological precursors of sgk1, and, where appropriate, a pharmaceutical excipient.

66. The pharmaceutical composition as claimed in claim 65, wherein said active compound is a polynucleotide which encodes a peptide, preferably a polypeptide, with this peptide influencing, preferably inhibiting or activating, the expression or function of activators, inhibitors, regulators or biological precursors of sgk1.

67. The pharmaceutical composition as claimed in claim 65, wherein said active compound is a small molecular compound, preferably a small molecular compound having a molecular weight (MW) of <1.000.
